(19) United States
(12) Patent Application Publication
(10) Pub. No.: US 2012/0289417 A1
(43) Pub. Date: Nov. 15, 2012

(54) T Cell Epitope Databases

(75) Inventors: Frank Carr, Babraham (GB); Matthew Paul Baker, Babraham (GB)

(73) Assignee: ANTITOPE LIMITED, Babraham (GB)

(21) Appl. No.: 13/562,854

(22) Filed: Jul. 31, 2012

Related U.S. Application Data
(62) Division of application No. 12/444,986, filed on Oct. 2, 2009.

(30) Foreign Application Priority Data
Oct. 11, 2006 (GB) 0620123.0
Oct. 11, 2006 (GB) 0620129.7

(51) Int. Cl. G01N 33/566; C40B 30/02

(52) U.S. Cl. 506/8; 435/7.24

(57) ABSTRACT

The invention relates to databases of T cell epitopes, especially helper T cell epitopes, for rapid interrogation of protein sequences for the presence of T cell epitopes. The invention includes full or partial databases and data structures of T cell epitopes including epitopes identified especially by ex vivo T cell assays with test peptides and includes T cell epitopes identified by extrapolation of data from test peptides. The present invention also includes high throughput methods for determining the T cell epitope activity of peptides for subsequent inclusion in databases and data structures including methods where subsets of T cell especially regulatory T cells are removed or inhibited from T cell assays in order to maximize the sensitivity of detection of T cell epitope activity.

The invention relates to databases of T cell epitopes, especially helper T cell epitopes, for rapid interrogation of protein sequences for the presence of T cell epitopes. The invention includes full or partial databases and data structures of T cell epitopes including epitopes identified especially by ex vivo T cell assays with test peptides and includes T cell epitopes identified by extrapolation of data from test peptides. The present invention also includes high throughput methods for determining the T cell epitope activity of peptides for subsequent inclusion in databases and data structures including methods where subsets of T cells especially regulatory T cells are removed or inhibited from T cell assays in order to maximize the sensitivity of detection of T cell epitope activity.

For pharmaceutical proteins administered to humans, immunogenicity manifested by the development of antibodies to the pharmaceutical protein is sometimes a limitation to the effectiveness and safety of the pharmaceutical protein in humans. In most cases, immunogenicity is likely to involve helper T cell epitopes which result from the presentation of peptides derived from the pharmaceutical protein on MHC class II and the subsequent activation of helper T cells by recognition of peptide-MHC class II complexes by T cell receptors on such T cells. Evidence for the involvement of helper T cell epitopes in immunogenicity includes clinical cases of immunogenicity where antibodies of the IgG isotype are detected suggesting helper T cell-induced Ig class switch. As such, T cell epitopes are considered to be important drivers of immunogenicity to pharmaceutical proteins and thus the measurement of such T cell epitopes in pharmaceutical proteins is highly desirable especially prior to testing in humans where the presence of such epitopes may be an important predictor of immunogenicity and therefore a factor in proceeding to such clinical trials or in the design of such trials.

Current methods for measurement of T cell epitopes include in silico methods, in vitro methods, ex vivo methods and in vivo methods. In silico methods typically relate to binding of peptides to MHC molecules and typically seek to mimic in vitro binding of peptides to MHC molecules. In silico methods range from those based on motifs of peptide sequences which bind MHC to methods involving computer modeling of peptide binding to MHC molecules. For MHC class II, in silico methods are largely restricted to HLA-DR where a homodimer of the DR molecule is involved in peptide binding. In silico methods for peptide binding to HLA-DQ and HLA-DP are generally much less accurate or not available due to the heterodomeric nature of DQ and DP binding and the more limited availability of in vitro MHC binding data. In vitro methods typically measure physical binding of peptides to MHC molecules typically using soluble or solubilised MHC molecules and labeled or tagged peptides. Ex vivo measurements typically use blood samples to measure helper T cell responses to peptides either by proliferation or by cytokine release. In vivo measurements typically use mice where either helper T cell responses to peptides are measured following injection of peptides or where subsequent antibody responses to the peptide are measured as an indirect indicator of helper T cell responses. In vivo measurements of non-murine T cell epitopes such as human T cell epitopes typically use either mice with reconstituted immune systems resultant from injection of human blood cells into SCID mice or mice which are transgenic for human MHC class II and which elicit T cell responses via presentation on human MHC class II.

Whilst in silico methods give potentially rapid prediction of binding of peptides to MHC class II, they do not accurately measure helper T cell epitopes which require other steps in addition to peptide-MHC binding including presence of non-tolerant T cells, T cell receptor recognition of peptide-MHC complexes, presence of specific cytokines and interaction of co-stimulatory molecules. Therefore in silico methods invariably over-predict the presence of T cell epitopes and, in addition, do not accurately predict HLA-DQ/DP restricted helper T cell epitopes. In addition, by predicting only MHC class II binding, in silico methods do not take account of the tolerance or non-responsiveness of T cells to certain MHC binding peptides, especially “self” peptides. Similarly, in vitro methods involving physical binding of peptides to MHC class II or binding of T cell receptors to peptide-MHC complexes do not take account of T cell tolerance or lack of T cell reactivity to peptide-MHC complexes. In addition, such methods are slow and do not provide measurement of T cell epitopes in real-time. Whilst ex vivo and in vivo methods provide the most stringent methods for measurement of T cell epitopes, these methods do not provide real time measurement of T cell epitopes and require specialist technical methods or specific animal strains. There is thus a need for new methods for measurement of T cell epitopes which are real time and simple to use.

The present invention relates to novel methods for measurement of T cell epitopes involving new databases and data structures of T cell epitopes derived from ex vivo or in vivo measurements. In particular, the invention relates to databases and data structures of actual T cell epitopes from ex vivo measurements whereby one or more, preferably all possible peptides which might occur in a test pharmaceutical protein have been previously tested for T cell epitope activity and whereby such measurement for each peptide is presented as a database or data structure for rapid interrogation of pharmaceutical protein sequences for the presence of T cell epitopes. As such, T cell epitopes in any pharmaceutical protein can be measured in real time without the need to run time-consuming technically specialist ex vivo measurements on peptides from the test pharmaceutical protein sequence. The present invention also includes methods for the enhanced detection of T cell epitopes by removal or inhibition of cellular subsets.

In a first aspect the present invention provides a method for determining if a test peptide sequence includes a T cell epitope by searching a database of sequences of peptides previously analysed for T cell epitope activity.

The database can be any database known to the skilled person, suitable for carrying out the invention. For example it can be a text file that can be searched using a BLAST program to identify similar sequences. The database can be part of a data structure. Any suitable data structure known to the skilled person can be used.

Preferably the database is searched for peptide sequences which are identical or share sequence similarity to the test peptide sequence.

The level of identity between two amino acid sequences sequences can be determined by aligning the sequences for optimal comparison purposes and comparing the amino acid residues at corresponding positions. The percent identity is determined by the number of identical amino acid residues in the sequences being compared (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The BLAST program of Altschul, et al. (1990) J. Mol. Biol. 215:403-410 have incorporated such an algorithm. When utilising BLAST and PSI-Blast programs, the default parameters of the respective programs can be used. See http://www.ncbi.nlm.nih.gov. Another example of a mathematical algorithm utilised for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

In the preferred method for establishing databases and data structures of helper T cell epitopes, multiple peptides representing multiple combinations of amino acids within a core MHC binding 9 amino acid sequence (‘core 9mer’) are tested in T cell assays (primarily human T cell assays) for induction of helper T cell responses, especially using T cell proliferation or cytokine release assay read-outs. Commonly, peptides of 10-15 amino acids in length will be tested which will include amino acids flanking either terminus of the core 9mer. Alternatively, 15mers with the same two amino acids flanking each terminus of the core 9mer will be tested, for example with two Alanine residues at each terminus. For a full analysis of all combinations of amino acid sequence within the core 9mer, 5.12×10¹¹ different combinations of amino acids in a 9mer (i.e. 20⁹) will be required. Thus one preferred method of the invention is to analyse all core 9mer sequences which have not been previously tested for helper T cell activity and to compile a helper T cell epitope database or data structure from all such analyses with, additionally, data from prior analysis of other core 9mers for helper T cell activity. Such a database or data structure will then allow users to rapidly analyse any specific core 9mer sequence for its helper T cell epitope activity.

In a derivative of the preferred method for establishing databases and data structures, a limited set of data for core 9mer T cell epitope activity will be analysed to identify partial sequences of amino acids which are associated with helper T cell epitope activity. Once such partial sequences are identified, sequences of additional potential helper T cell epitopes can be extrapolated and entered into the database and data structure along with sequences for actual T cell epitopes used to identify the partial sequences. For example, it is recognized that within the core 9mer of a helper T cell epitope, amino acids at position 1, 4, 6, 7 and 9 are primarily involved in binding to MHC class II leaving amino acids 2, 3, 5 and 8 as the main amino acids which interface with the T cell receptor. Therefore, sets of data can be obtained for MHC binding peptides with fixed residues at positions 1, 4, 6, 7 and 9 and variations in amino acids restricted to positions 2, 3, 5 and 8 thus requiring only 160,000 peptides with core 9mer sequence of FXXFXFFXF, where F=a fixed amino acid residue and X=a variant residue comprising any of the 20 natural amino acids in all possible combinations. Exclusion of certain peptide sequences which are known not to result in helper T cell activity (such as where each X=Proline) and sequences of X's already known not to induce helper T cell activity will reduce the number of peptides which are required to be tested. Alternatively or additionally, exclusion of 9mer sequences with position 1 which is not hydrophobic (hydrophobic=Ala, Ileu, Leu, Met, Phe, Val) will also reduce the number of peptides which are required to be tested.

In the preferred method of the present invention, one or more test peptide sequences will be analysed by searching a database or data structure for identical or similar peptides which have been previously analysed for helper T cell activity. Typically peptides of length 9-15 amino acids, preferably 9 amino acids will be analysed by searching the database for identical or similar peptides. This will include identifying peptides with identical 9mer sequences, or for peptides with homology to the test peptide (typically with 5 or more amino acids at corresponding relative positions within the test and database peptide sequences). In one preferred embodiment peptides with identical or similar amino acids at corresponding relative 1, 4, 6, 7 and 9 positions within the test peptide sequence and the peptide sequences in the database or data structure will be identified. Alternatively, peptides with identical or similar corresponding relative 2, 3, 5 and 8 positions within the test peptide sequence and the peptide sequences in the database or data structure will be detected. For example, a test 9 amino acid peptide with a sequence ADEFGHIKL may be considered a possible T cell epitope if a T cell epitope sequence in the database is composed of (or includes) AAAFAHIAL (i.e. corresponding relative 1, 4, 6, 7 and 9 positions) or ADEAGAAKA (i.e. corresponding relative 2, 3, 5 and 8 positions). Typically, such analysis of peptides especially those with corresponding relative 2, 3, 5 and 8 positions will also include a separate analysis of the putative core 9mer MHC binding, commonly using in silico methods or in vitro methods such that the possible T cell epitope identified will be excluded if there is no significant binding to MHC. For example, whilst a test 9 amino acid peptide with a sequence GDEFGHIKL will be matched with the database peptide ADEAGAAKA with corresponding relative 2, 3, 5 and 8 positions, this peptide will likely be excluded as a T cell epitope due to the absence of a hydrophobic amino acid at position 1 or a lack of MHC binding following in silico or in vitro measurement of peptide-MHC binding.

The present invention will include methods for obtaining data for inclusion in the database or data structure and typically will involve analysing peptides individually for helper T cell epitope activity using standard ex vivo helper T cell assay formats such as the Elispot format where cytokine release from helper T cells is measured. Typically such assay formats limit the number of peptides which can be practically tested in one experiment usually to <500 peptides and also limit the sensitivity of detection of T cell epitopes in peptides. Potentially such assay formats can be reconfigured or miniaturized to greatly enhance peptide throughput, for example by testing pools of peptides for induction of helper T cells and thereafter de-replicating such pools for individual peptides which induce helper T cells, or by using microformats where high densities of peptides or cells are tested simultaneously, for example in arrays of peptides previously synthesised on pins, and where highly sensitive assays for T cell proliferation and cytokine release are adapted for such high density assays. Alternatively, rather than using high density arrays of peptides or arrays of cells for testing different peptides, ex vivo T cell assays can be performed in fluid microdroplets whereby peptides react with cells inside a microdroplet whereby such microdroplets can be analysed individually, for example by FACS (fluorescence activated cell sorting) using, for example, a fluorometric measurement of cytokine release or incorporation of fluorescinated tracer into proliferating T cells such as fluorescein-labeled BUDR (5′-bromodeoxyuridine). Other assay formats will include assays where individually activated helper T cells can be detected and the activating peptide sequence determined. Such assays formats may be facilitated by the availability of MHC class II tetramers where individual peptides or groups of peptides can be bound to MHC class II with tetramers and then tested for activation of T cells such that the activating peptides can subsequently be identified including, for groups of peptides synthesized semi-randomly, by tags associated with the activating peptide or by direct identification of the activating peptide by mass spectrometry.

For all of the aforementioned assay formats, the invention includes improvement in sensitivity of detection of T cell epitopes by removal of cellular subsets, especially subsets of T cells and especially removal of regulatory T cells from T cell assay mixtures which results in substantial increases in helper T cell responses to test antigens. Thus in a second aspect the invention provides a method for creating a database of helper T cell responses to a test substance comprising the follows steps;

(a) isolating antigen-presenting cells (APCs) and T cells from an organism
(b) depleting or inhibiting regulatory T cells from the isolated cells
(c) incubating said regulatory T cell-depleted cells with the test substance
(d) measurement of T cell responses to the test substance

Thus, the present invention also includes novel T cell assay methods for optimal detection of T cell epitopes where regulatory T cells are removed from cultures resulting in an increase in T cell responses to test antigens. In particular, regulatory T cell are removed by removal of T cells expressing high levels of surface CD25 antigen (CD25hi T cells), preferably where methods are employed which remove, inhibit or destroy between 5 and 75% of CD25hi T cells and, in particular, between 10 and 25% CD25hi T cells.

The APCs and T cells are normally obtained from a blood sample. However, different sources of T cells and/or APCs can be used in the invention including those derived from tonsils, Peyer's Patch, tumours and cell lines. In one preferred embodiment, the method is carried out using human peripheral blood mononuclear cells (PBMCs).

As used herein the term “depleting” means elimination of some of the regulatory T cells. This can be done by physically removing the cells or by inhibiting or modulating the action of the T cells. Thus the activity of the targeted T cells is reduced.

It will be understood by those skilled in the art that, as part of the present invention, a range of methods for the depletion or targeting of regulatory T cells might be used as alternatives to the depletion of regulatory T cells by virtue of CD25^{hi}. It will also be understood that the present invention will also include methods for modulation of the effects of regulatory T cells in T cell assays. For depletion or targeting, molecules expressed on the surface of regulatory T cells may be used in conjunction with or as alternatives to CD25 for the depletion of these cells. Such molecules may include but not be limited to GITR, CTLA-4, CD103, CC chemokine receptor 4, CD62L and CD45RA and may also include surface-associated cytokines or surface forms of cytokines such as IL-10 and TGFβ. Depletion may be achieved by several methods including binding to specific antibodies to adsorb regulatory T cells onto a solid phase, or to cause the destruction or inhibition of such regulatory T cells, or otherwise to separate regulatory T cells from other T cells for the T cell assays. For modulation, molecules secreted by regulatory T cells may be prevented from such secretion or may be blocked/inhibited/destroyed after secretion. Such molecules may include cytokines such as IL-10, IL-4, IL-5 and TGFβ and such molecules may be blocked using organic or inorganic molecules which bind to such molecules, for example antibodies or soluble receptors, or by inhibitory nucleic acids such as siRNA, antisense oligonucletides, or other nucleic acids delivered into regulatory T cells or induced within such cells. Modulation of regulatory T cell activity may also be achieved by targeting receptors or other surface molecules on regulatory T cells including but not limited to GITR, CTLA-4. CD103, CC chemokine receptor 4, CD62L and CD45RA in such a way as to break the suppressive function of these cells. Such inhibition of function may be achieved, for example, by specific antibodies with an agonist function or which may block ligand-target interactions such that regulatory T cells are not removed but are rendered non-functional. Modulation of regulatory T cell activity may also be achieved by blocking the target receptors of molecules secreted by regulatory T cells or by blocking pathways activated or down-regulated by such secreted molecules. Also for modulation, regulatory T cells may be inhibited directly, for example by blocking of transcription factors such as foxp3 or blocking of other functions or pathways related to regulatory T cells. Such inhibition or blocking may be achieved by organic or inorganic molecules, or by inhibitory nucleic acids such as siRNA, antisense oligonucletides, or other nucleic acids delivered into regulatory T cells or induced within such cells. In all cases where organic, inorganic or nucleic acid molecules are used to inhibit the action of or otherwise modulate regulatory T cells, where such molecules themselves interfere with T cell assays, such molecules will preferably be removed from such assays or modified to a form which will not interfere with such assays. For example, specific antibodies or proteins used to remove molecules secreted by regulatory T cells will either be selectively removed prior to T cell assays or will be used in a specific form which will not interfere with T cell assays. For example, for human T cell assays, a human form of an antibody or protein will be used to avoid T cell responses to the antibody or protein itself.

Preferably, the assay method is used with human peripheral blood mononuclear cells (PBMCs) with key steps as follows;
(1) PBMCs are isolated from human blood samples
(2) CD8⁺ T cells are removed
(3) CD25^{hi} T cells are depleted
(4) Cultures are incubated with test antigens at one or more concentrations and tested at one or more time points for T cell proliferation and/or cytokine release

Measurements of T cell epitope activity in the present invention can relate to T cell epitope activity in relation to single MHC allotypes or to multiple MHC class II allotypes. Thus individual peptides can be tested with either single or multiple MHC allotypes and databases can therefore relate either to single or multiple MHC allotypes. In the preferred method of the present invention, peptides are tested with multiple MHC allotypes, for example for human helper T cell epitopes, peptides would typically be tested with at least 20 different MHC-typed human blood samples (and typically 40-60 blood samples) and MHC association of active peptides determined from such MHC-typing of the samples. In the preferred method of the invention, T cell epitope databases and data structures will be annotated with data on associations with MHC allotypes. In addition, T cell epitope databases may be annotated with details of the donor and, for peptides containing T cell epitopes, details of the T cell responses such as data relating to primary or secondary responses, proliferation and cytokine measurements, percentage of donors responding, magnitude of responses, and full MHC types of donors responding.

Irrespective of the methods used for determining the T cell epitope activity of multiple peptides, the current invention discloses databases and data structures of T cell epitopes (primarily helper T cell epitopes) especially for rapid interrogation of pharmaceutical protein sequences for the presence of T cell epitopes. Such T cell epitope databases and data structures may be derived from testing of multiple individual peptides for T cell epitope activity or from entering other data including all known T cell epitopes. Such databases and data structures may comprise data from complete sets of peptides or incomplete sets of peptides such that data will not be available for some peptides tested by interrogation of the database. The current invention also includes, in addition to the concept of databases and data structures, novel methods for testing multiple peptides for inclusion in such databases and data structures, especially methods for determining helper T cell epitope activity of multiple peptides.

A particular use of the present invention will be to analyse proteinaceous pharmaceuticals for the presence of T cell epitopes, especially helper T cell epitopes. This will be particularly useful for determining the immunogenicity or vaccine potential of such pharmaceuticals, measured by the presence of T cell epitopes and other factors such as the frequency and magnitude of T cell responses, and the donor MHC association of such responses. The invention will be especially useful in pharmaceutical research where the immunogenicity of different protein variants can be determined by analysis of their protein sequences by the methods of the invention. For pharmaceutical use, proteins variants with lowest frequency of T cell epitopes will commonly be selected as leads with lowest potential for immunogenicity.

A further use of the present invention will be in the creation of novel proteinaceous pharmaceuticals either for therapeutic or vaccine use. For therapeutic use, methods of the present invention will be used to create novel protein variants derived from a starting protein wherein the number of T cell epitopes is reduced or the T cell epitopes are removed in such variants. Typically, therapeutic protein variants will be generated by replacing sequences in the starting protein with new sequences from the database with no T cell epitope activity, whereby such replacement does not create new T cell epitopes through combinations of sequences from the starting protein and database peptide, or by combinations of sequences from database peptides. For vaccine use, methods of the present invention will be used to create novel protein variants derived from a starting protein wherein the number of T cell epitopes increased in such variants. A particularly useful method of the present invention will be to generate novel improved protein variants which retain the desirable properties of starting proteins but which also include improved properties such as potentially reduced immunogenicity through a reduction or elimination of T cell epitopes.

Such a method will typically involve the following key steps;
(a) analysis of one or more existing proteins to determine amino acids (‘desirable residues”) required to provide desirable properties in a new protein;
(b) selection from the peptide sequence database of one or more peptides containing said desirable residues for inclusion in the improved protein at positions corresponding to those in the existing protein whereby such peptides are not T cell epitopes;
(c) synthesis of the improved protein by inclusion of one or more said selected peptides.

For vaccine use, a particularly useful method of the present invention will be to generate novel improved protein variants which retain desirable properties of starting proteins but which also include additional T cell epitopes. Such method will typically involve the following key steps;
(a) analysis of one or more existing proteins to determine amino acids (‘desirable residues”) required to provide desirable properties in a new protein;
(b) selection from the peptide sequence database of one or more peptides containing said desirable residues for inclusion in the improved protein at positions corresponding to those in the existing protein whereby some or all of such peptides include T cell epitopes;
(c) synthesis of the improved protein by inclusion of one or more said selected peptides.

As used herein an “improved protein variant” is a protein which has been adapted to either increase or reduce the potential immungenicity of the protein, depending on its intended use, whilst maintaining the desirable properties of the protein. For example, a protein which is suitable for therapeutic used can be improved, by removing any T cell epitopes which may cause an adverse reaction. Alternatively, a protein which is suitable for use as a vaccine may have further T cell epitopes added to increase the potential immune response, and thus increase the protective effect provided.

As used herein “desirable properties” refers to the properties of a protein which are required for the protein to maintain its required function. For example for therapeutic proteins this could be the ability to inhibit the activity of a target molecules, such as an enzyme. Alternatively the desirable properties could be attributed to the parts of the protein which increase the half-life of the protein in the blood. In addition for proteins used as vaccines, the epitopes which induce the immungenic response should be retained.

It will be understood by those skilled in the art that the present invention includes any database or data structure of T cell epitopes irrespective of the source of the measurement of T cell epitope activity. It will be understood that databases and data structures of the present invention relate to T cell epitopes identified in assays employing living T cells such as ex vivo T cell assays or T cell assays from in vivo studies, for example studies where peptides are injected into an organism and measurements of activity on live T cells undertaken. It will be understood that databases and data structures of the present invention will include data on active T cell epitopes as well as on peptides with no effects of T cells. It will be understood that such databases or data structures may be partial databases where data on certain sequences of peptides is not included. Alternatively they can be complete databases or data structures including all possible sequences of peptides of a certain length, typically 9mers for helper T cell epitopes with, typically, flanking amino acids at the N and/or C termini of the peptide. It will be understood that databases and data structures of the present invention will relate to T cell epitopes, preferably of helper T cell type associated with MHC class II, but also MHC class I restricted epitopes, especially cytotoxic T cell epitopes. Databases and data structures of the present invention may also comprise or consist of peptides with other activities on T cells such as peptides which stimulate regulatory T cells and peptides which directly down regulate or inhibit T cells.

The invention will be illustrated but not limited by the following examples. The following examples should not be considered limiting for the scope of the invention. The figures and tables relate to the examples below and are as follows;

Table 1: shows the results of T cell proliferation assays of peptides with fixed T cell receptor contact residues derived from a T cell epitope on a background of various MHC contact residues from other T cell epitopes (cf example 3).

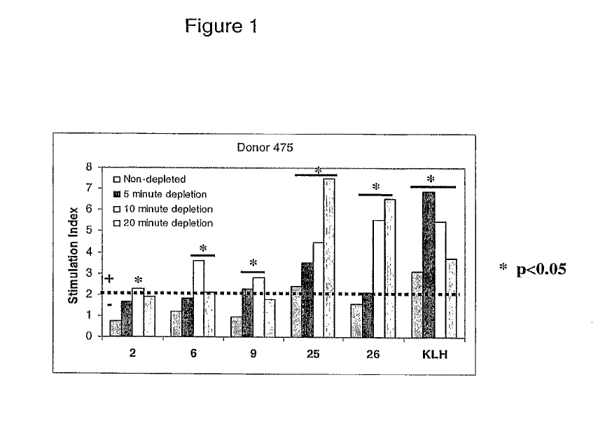

FIG. 1: shows the effect of depletion of CD25hi T cells on helper T cell responses (Stimulation Index=ratio of T cell proliferation with:without peptide) after addition of various peptides or KLH (cf example 1).

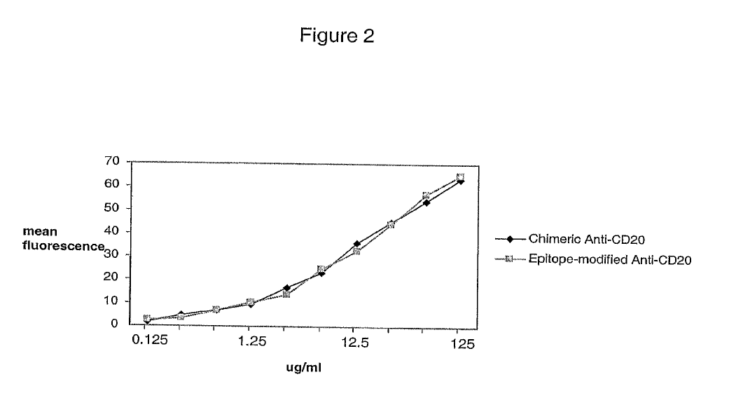

FIG. 2: shows the results of a FACS analysis of the binding of serial dilutions of chimeric anti-CD20 antibody and epitope-modified antibody where T cell epitopes identified by T cell assays were replaced by selection of database peptide sequences for non-T cell epitopes (cf example 4).

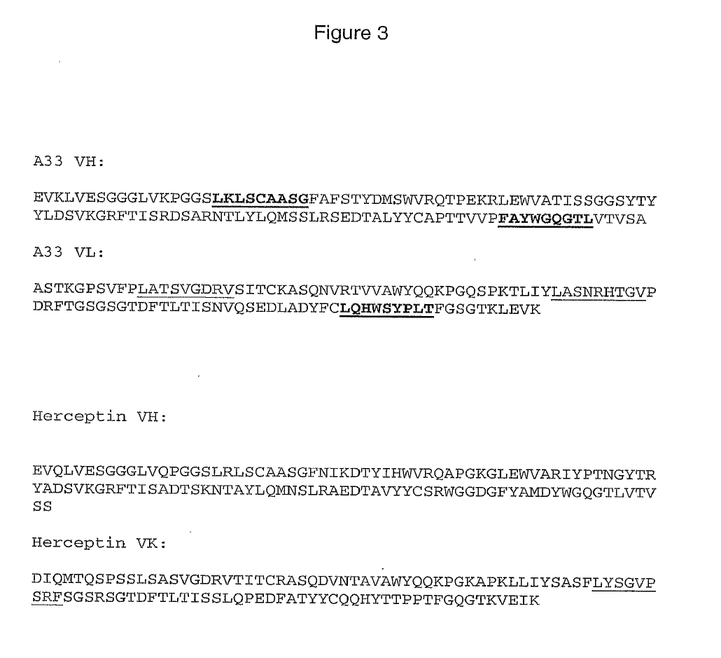

FIG. 3: shows a comparative analysis of variable region sequences of humanized A33 and anti-HER2 antibodies by searching the T cell epitope database for identical matched T cell epitope core 9mers and MHC binding 9mers with relative corresponding 2, 3, 5 and 8 residues (cf example 5).

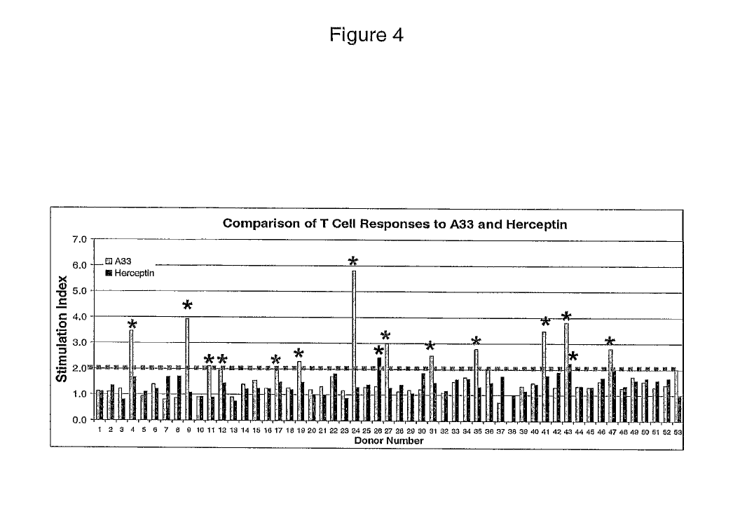

FIG. 4: shows a T cell assay of whole humanized A33 and anti-HER2 antibodies (cf example 5).

### EXAMPLE 1

### Method for Determining T Cell Epitopes and Generation of a T Cell Epitope Database

Peripheral blood mononuclear cells were isolated from healthy community donor buffy coats (from blood drawn within 24 hours) obtained from National Blood Transfusion Service (Addenbrooke's Hospital, Cambridge, UK) and according to approval granted by Addenbrooke's Hospital Local Research Ethics Committee. PBMC were isolated from buffy coats by Ficoll (GE Healthcare, Chalfont St Giles, UK) density centrifugation and CD8+ T cells were depleted using CD8+ RossetteSep™ (StemCell Technologies, Vancouver, Canada). Donors were characterized by identifying HLA-DR haplotypes using an Allset™ SSP-PCR based tissue-typing kit (Dynal, Wirral, UK) as well as determining T cell responses to a control antigen Keyhole Limpet Haemocyanin (KLH) (Pierce, Cramlington, UK), Tetanus Toxoid (Aventis Pasteur, Lyon, France) and control peptide epitope from Influenza HA (C32, aa 307-319).

CD25^{hi} T cell depletion was carried out using anti-CD25 Microbeads from Miltenyi Biotech (Guildford, UK) using the supplier's standard protocol and magnet. 10 vials of each donor was thawed and cells were resuspended in 30 mls 2% inactivated human serum/PBS (Autogen Bioclear, Caine, Wiltshire, UK). 5×10⁷ cells were transferred to 3×15 ml tubes with the remaining cells kept as whole PBMCs. An anti-CD25 microbeads dilution mixture was made using 300 μl of beads+4200 μl of separation buffer (0.5% human serum/2 mM EDTA/PBS). The 15 ml tubes were centrifuged and resuspended in 500 μl of microbeads dilution mixture. Tubes were then kept at 4° C. for 5, 10 or 20 minutes before separating on the column. Columns were set up by placing column in the magnet supported on a stand, adding 2 mls separation buffer to column and allowing it to drip through. After incubation with beads 10 ml separation buffer was added and tubes were centrifuged at 1500 rpm for 7 minutes. Cells were then resuspended in 500 μl of separation buffer and added to the column followed by 2×1 ml washes with separation buffer. The flow through the column was collected in 15 ml tubes and contained the CD25^{hi} T cell depleted fraction. These cells were spun down at 1500 rpm for 7 minutes and resuspended in 3 ml AIMV medium (Invitrogen, Paisley, UK) before counting.

Cells were stained for CD4 and CD25 and cell numbers detected by FACS. 5-10×10⁵ cells of each cell population were put in one well of a 96-well U bottomed plate (Greiner Bio-One, Frickenhausen, Germany). The plate was spun down at 1200 rpm for 4 minutes. Supernatant was ejected and cells were resuspended in 50 μl antibody dilution. Antibody dilution consisted of 1/50 dilution of FITC-labeled anti-CD4 antibody (R&D Systems, Minneapolis, USA)+1/25 dilution of PE-labeled anti-CD25 antibody (R&D Systems, Minneapolis, USA) in FACS buffer (1% human serum/0.01% Sodium azide/PBS). Control wells were also unstained, stained with isotype controls or single stained with labeled antibody.

Plates were incubated on ice for 30 minutes in the dark. Plates were then spun down at 1200 rpm for 4 minutes. Supernatant was ejected and cells were resuspended in 200 μl FACS buffer. This was repeated twice and cells were then transferred to FACS tubes. Cells were run through a FACS Calibur (Becton Dickinson, Oxford, UK), and data collected and analysed based on size, granularity and fluorescent tags.

Proliferation assays were carried out as follows. Whole CD8⁺ T cell depleted PBMC and CD8⁺ CD25^{hi} depleted PBMC were added at 2×10⁵ per well in 100 μl of AIMV. Using flat bottom 96 well plates, triplicate cultures were established for each test condition. For each peptide 100 μl was added to the cell cultures to give a final concentration of 5 μM. Cells were incubated with peptides and protein antigens for 7 days before pulsing each well with 1 mCi/ml 3HTdR (GE Healthcare, Chalfont St Giles, UK), for 18 hours.

For the proliferation assay, a threshold of a stimulation index equal to or greater than 2 (SI≧2) was used whereby peptides inducing proliferative responses above this threshold were deemed positive (dotted line). All data was analysed to determine the coefficient of variance (CV), standard deviation (SD) and significance (p<0.05) using a one way, unpaired Student's T test. All responses shown with SI≧2 were significantly different (p<0.05) from untreated media controls.

The results are shown in FIG. 1 which represent T cell proliferative responses in PBMCs from one of the human donors tested (donor 475) to a series of borderline or weak T cell epitopes (peptides 2 (GDKFVSWYQQGSGQS), 6 (IKPEAPGCDASPEELNRYYASLRHYLNLVTRQRY), 9 (QSISNWLNWYQQKPG)) and to a pair of strong T cell epitopes (peptides 25 (PKYRNMQPLNSLKIAT) and 26 (TVFYNIPPMPL)) and to KLH antigen. The results show an increase in T cell responses for all peptides after depletion of CD25^{hi} T cells. Maximum responses were determined for all peptides following 10 or 20 minute depletion of CD25^{hi} T cells. These results demonstrated strong increases in T cell responses after CD25^{hi} T cell depletion which, in the examples of peptides such as peptides 2 and 9, allowed detection of T cell epitopes in peptides previously scored borderline or negative for T cell responses.

Mutations in the above peptides 2, 9, 25 and 26 were made as follows;

```
  2-F→G (GDKGVSWYQQGSGQS)
  9-L→G (QSISNWGNWYQQKPG)
25 M→G (PKYRNGQPLNSLKIAT)
26 F→G (TVGYNIPPMPL)
```

These peptides were retested in the proliferation assays as above including CD25^{hi} T cell depletion for 10 and 20 minutes and including donor 475. No donors including donor 475 gave a significant T cell response to any of these mutated peptides. Thus peptides 2, 6, 9, 25 and 26 were entered into the database as helper T cell epitopes whilst peptides 2-F→G, 9-L→G, 25 M→G and 26 F→G were entered as negative for helper T cell epitope responses. Parallel analysis of the non-mutated peptides sequences by the TEPITOPE method of Sturniolo et al. (Nature Biotechnology, vol 17 (1999) p555-561) indicated that the likely P1 positions for MHC class II binding by these peptides were at the amino acids which were subsequently mutated to G (glycine) residues and thus these peptides were annotated in the database with the putative residues in the core MHC binding 9mer including the amino acids at the relative 1, 4, 6, 7 and 9 positions for MHC class binding, and the amino acids at 2, 3, 5 and 8 positions for T cell receptor recognition.

### EXAMPLE 2

### Analysis of Peptides with Fixed MHC Contact Residues

The following peptides with fixed relative 1, 4, 6, 7 and 9 positions were analysed using (i) a database of T cell epitopes generated using the method of example 1, (ii) the TEPITOPE algorithm for peptide-MHC binding prediction (Sturniolo et al., ibid), and (iii) the T cell assay method of example 1:

```
  1-NWLRNYDQKQGAT
  2-NWLEGYHQKIGAT
  3-NWLLKYMQKFGAT
  4-NWLPSYTQKWGAT
  5-NWLYVYAQKRGAT
  6-NWLNDYQQKEGAT
  7-NWLGHYIQKLGAT
  8-NWLKMYFQKPGAT
  9-NWLSTYWQKYGAT
10-NWLAAYAQKAGAT
11-NWGRNYDQKQGAT
12-NWGEGYHQKIGAT
13-NWGLKYMQKFGAT
14-NWGPSYTQKWGAT
15-NWGYVYAQKRGAT
16-NWGNDYQQKEGAT
17-NWGGHYIQKLGAT
18-NWGKMYFQKPGAT
19-NWGSTYWQKYGAT
20-NWGAAYAQKAGAT
```

Peptides 1-10 all included a three amino acid N-terminal sequence of NWL whilst peptides 11-20 were analogues of peptides 1-10 except that the third N-terminal amino acid was G instead of L. Interrogation of the T cell epitope database identified, for peptides 1 to 10 above, a previous helper T cell epitope with identical corresponding relative positions 1, 4, 6, 7 and 9 in the peptide QSISNWLNWYQQKPG corresponding to peptide 9 in example 1 whereby previous TEPITOPE analysis had indicated a MHC binding core 9mer of LNWYQQKPG. Peptides 11 to 20 lacked the important hydrophobic P1 anchor in the core 9mer and thus were provisionally scored as non-epitopes. This analysis was supported by TEPITOPE analysis of peptides 1 to 20 which predicted that peptides 1 to 10 but not 11-20 bound to a range of MHC class II allotypes.

Analysis of peptides 1-20 using the T cell assay method of example 1 and using donor 475 (cf FIG. 1) demonstrated that peptides 1 to 6 and 8 to 10 gave significant helper T cell responses whilst peptides 7 and 11-20 gave no significant responses. This indicated that the database match with peptide 9 from example 1 had resulted in correct identification of previously unanalysed peptides 1 to 6 and 8 to 10 (with common relative 1, 4, 6, 7 and 9 positions) as T cell epitopes. Further interrogation of the database for matches at corresponding relative positions 2, 3, 5 and 8 identified a peptide sequence GFGHEIGPLGEP which was previously scored by T cell assays as a non-T cell epitope and which had identical 2, 3, 5 and 8 positions to peptide 7 (NWLGHYIQKLGAT) (and also peptide 17 (NWGGHYIQKLGAT)). This indicated that these T cell receptor contact residues, within a peptide which bound to MHC class II, did not result in a T cell response. This indicated that the database match of peptides 7 and 17, with a non-T cell epitope peptide with identical residues at corresponding relative positions 2, 3, 5 and 8, resulted in correct identification of previously unanalysed peptides 7 and 17 as non-T cell epitopes. Overall, this example demonstrated potential T cell epitope activity of test peptides with matching common relative 1, 4, 6, 7 and 9 positions to a known T cell epitope although information from peptides with corresponding relative positions 2, 3, 5 and 8 can determine whether the test peptide contained a T cell epitope or not.

### EXAMPLE 3

### Analysis of Peptides with Fixed T Cell Receptor Contact Residues

The ability of constant T cell receptor contact residues (corresponding relative positions 2, 3, 5 and 8) to induce T cell responses on a background of any combination of MHC contact residues (corresponding relative positions 1, 4, 6, 7 and 9) in an MHC binding peptide was tested using the T cell receptor contact residues from a confirmed database T cell epitope with a core 9mer LQHWSYPLT. The T cell receptor contact residues _QH_S_L were substituted onto a background of four other database T cell epitopes as follows; FLLTRILTI, ILWEWASVR, LSCAAGGRA and FKGEQGPKG resulting in the test peptides FQHTSILLI, IQHESASLR, LQHASGGLA and FQHESGPLG. Control peptides were also made with altered P1 residues (F->G) as follows; GQHWSYPLT, GQHTSILLI, GQHESASLR, GQHASGGLA and GQHESGPLG.

These peptides were tested by the T cell assay method of example 1 using 50 donors with a range of MHC class haplotypes. The number of responding donors (from 50) and the mean stimulation index (SI) for responding donors were measured and compared for the test peptides. The results are shown in Table 1 and demonstrate that the fixed T cell receptor contact residues _QH_S_L could trigger helper T cell responses on each of the different background MHC contact residues from four other T cell epitopes and that such T cell responses were eliminated if MHC binding was eliminated by elimination of the hydrophobic P1 residue. This example also demonstrates the potential for creating a large database of peptides with known T cell epitope activity by testing all combinations of possible T cell receptor contact residues at corresponding relative positions 2, 3, 5 and 8 on a fixed background of MHC binding residues, thus requiring analysis of only 20⁴ peptides (160,000) in T cell assays.

### EXAMPLE 4

### Generation of a Variant Anti-CD20 Antibody by T Cell Epitope Removal

The database of T cell epitopes was used to identify known T cell epitopes in the anti-CD20 antibody Leu16 (Gillies et al., Blood 105 (2006) p3972-39′78). Overlapping 15mers starting from the N-terminus of the Leu16 heavy chain variable region (VH) sequence 5′-EVQLQQSGAELVKPGASVKMSCKASGYTFTSYNMHWVKQTPGQGLEWIGAIYP GNGDTSYNQKFKGKATLTADKSSSTAYMQLSSLTSEDSADYYCARSNYYGSSY WFFDVWGAGTTVTVSS-3′ together with overlapping 15mers starting from the N-terminus of the Leu16 light chain variable region (VL) sequence 5′-DIVLTQSPAILSASPGEKVTMTCRASSSVNYMDWYQKKPGSSPKPWIYATSNLAS GVPARFSGSGSGTSYSLTISRVEAEDAATYYCQQWSFNPPTFGGGTKLEIK-3′ were analysed resulting in the identification of 3 actual T cell epitopes (identical core 9mer) in the VH and two potential T cell epitopes (identical residues at corresponding relative positions 2, 3, 5 and 8 with a hydrophobic P1 anchor) in VL as follows;

| | | Database |
|---|---|---|
| | | Epitope 9 mer |
| Leu16VH | LVKPGASVK | LVKPGASVK |
| | FKGKATLTA | FKGKATLTA |
| | LTSEDSADY | LTSEDSADY |
| Leu16VL | ILSASPGEK | LLSGSPAEK |
| | MDWYQKKPG | LDWYQKKPG |

Recombinant DNA techniques were performed using methods well known in the art and, as appropriate, supplier instructions for use of enzymes used in these methods. Sources of general methods included Molecular Cloning, A Laboratory Manual, 3^{rd} edition, vols 1-3, eds. Sambrook and Russel (2001) Cold Spring Harbor Laboratory Press, and Current Protocols in Molecular Biology, ed. Ausubel, John Wiley and Sons. The Leu16 variable region genes were cloned and modified using the methods of Gillies et al., ibid to introduce new peptide sequences to replace the above T cell epitope-related core 9mers. Compatible non-epitope 9mer peptides were selected from the database as follows;

| | Putative T | | Database Non- |
|---|---|---|---|
| | cell epitope | | Epitope 9 mer |
| Leu16VH | LVKPGASVK | [symbol] | VVKPGASVK |
| | FKGKATLTA | [symbol] | FKGRVTLTA |
| | LTSEDSADY | [symbol] | LRSEDSAVY |
| Leu16VL | ILSASPGEK | [symbol] | TLSASPGEK |
| | MDWYQKKPG | [symbol] | MAWYQQKPG |

These modified 9mers were introduced into the Leu VH and VL sequences by PCR and the resultant genes cloned into separate vectors providing human IgG1 and human κ constant regions to encode chimeric heavy and light chains respectively. Plasmids containing unmodified (chimeric) and epitope modified Leu16 heavy and light chains were transfected into NSO cells and stable transformants were selected for antibody harvesting and purification using Protein A.

Testing of the antibodies was performed according to Gillies et al., ibid, and used the CD20+ human Daudi Burkitt lymphoma cell line (ATCC, Rockville, Md.) was used as a target. Binding assays were performed in a FACS format for testing binding of chimeric anti-CD20 in comparison to the modified antibody with inserted database non-epitopes. The results (FIG. 2) show that the epitope modified anti-CD20 antibody derived from Leu 16 binds with similar efficiency to Daudi cells compared to chimeric anti-CD20. The epitope modified anti-CD20 provides for a potentially less immunogenic alternative to the chimeric anti-CD20 antibody.

### EXAMPLE 5

### Comparison of A33 and Anti-HER2 Antibody Variable Regions for Presence of T Cell Epitopes

Sequences of the variable regions of two humanised antibodies, the humanised A33 antibody (U.S. Pat. No. 6,307,026, Celltech Ltd.) and the humanised anti-HER2 antibody known as Herceptin® (Carter et al., Proc. Nat. Acad. Sci. USA, vol 89 (1992) p4285, U.S. Pat. No. 5,821,337) were compared by searching the T cell epitope database. The database of T cell epitopes generated according to example 1 was searched for identical 9mer sequences and also for 9mers with corresponding relative positions 2, 3, 5 and 8. The results are shown in FIG. 3. For humanised A33, three identical 9mers from peptides positive for T cell epitope activity were identified in the database together with two matches with epitopes with corresponding relative positions 2, 3, 5 and 8 where the core 9mer from humanised A33 was predicted according to Sturniolo et al., ibid to bind MHC class II. A range of matches were found with database peptides with no T cell epitope activity (not shown). For humanised anti-HER2 antibody, no identical 9mers from peptides positive for T cell epitope activity were identified in the database and a single match with an epitope with corresponding relative positions 2, 3, 5 and 8 was identified where the core 9mer was predicted to bind MHC class II.

The humanised A33 and anti-HER2 antibodies were constructed according to the methods of example 4. These were analysed in the T cell assays as in example 1 using 53 donors in proliferation assays and were performed by adding 1 ml of antibody to a final concentration of 10 μg/ml. The data in FIG. 4 shows the maximum stimulation index between days 5 and 8 after antibody addition and indicates that significant T cell responses were observed for 13 out of 53 donors to humanised A33 and only 2 out of 53 donors to humanised anti-HER2 antibody.

These data indicate that the variable region of the humanised A33 antibody contains significant T cell epitopes (three actual, three predicted) whilst the humanised anti-HER2 antibody contains no confirmed T cell epitopes and only one predicted epitope with a pre-determined motif at positions 2, 3, 5 and 8 from another epitope. These data also are consistent with the lower level of clinical immunogenicity of the humanised anti-HER2 antibody (Herceptin®) compared to humanised A33.

TABLE 1

| | Number of | |
|---|---|---|
| | responding donors | Mean SI |
| LQHWSYPLT | 5 | 6.3 +− 1.3 |
| FLLTRILTI | 6 | 3.9 +− 1.6 |
| ILWEWASVR | 3 | 2.6 +− 0.6 |
| LSCAAGGRA | 3 | 2.3 +− 0.5 |
| FKGEQGPKG | 4 | 3.5 +− 0.7 |
| FQHTSILLI | 5 | 3.8 +− 0.7 |
| IQHESASLR | 3 | 2.6 +− 0.1 |
| LQHASGGLA | 2 | 2.2 +− 0.2 |
| FQHESGPLG | 3 | 2.8 +− 0.7 |
| GQHWSYPLT | 0 | — |
| GQHTSILLI | 0 | — |
| GQHESASLR | 0 | — |
| GQHASGGLA | 0 | — |
| GQHESGPLG | 0 | — |

1.-31. (canceled)

32. A method for detection of helper T cell epitopes in a test protein sequence by analysis of 9mers from the test protein sequence by both of the following steps;
(a) Analysis of known helper T cell epitopes and identification of identical matches for residues 2, 3, 5 and 8 between one or more T cell epitopes and a 9mer sequence in the test protein sequence;
(b) Analysis and detection of binding by the same 9mer sequence of the test protein to MHC class II molecules.

33. The method of claim 32 where matches with known helper T cell epitopes are determined by searching a database of helper T cell epitopes.

34. The method of claim 32 wherein the helper T cell epitopes and MHC class II molecules are both human.

35. The method of claim 32 where detection of binding of 9mer sequences from the test protein to MHC class II molecules is determined by in silico analysis of the 9mer for binding to MHC class II.

36. The method of claim 33 where detection of binding of 9mer sequences from the test protein to MHC class II molecules is determined by in silico analysis of the 9mer for binding to MHC class II.

37. The method of claim 34 where detection of binding of 9mer sequences from the test protein to MHC class II molecules is determined by in silico analysis of the 9mer for binding to MHC class II.

38. The method of claim 32 where detection of binding of 9mer sequences from the test protein to MHC class II molecules is determined by in vitro analysis of the 9mer for binding to MHC class II.

39. The method of claim 33 where detection of binding of 9mer sequences from the test protein to MHC class II molecules is determined by in vitro analysis of the 9mer for binding to MHC class II.

40. The method of claim 34 where detection of binding of 9mer sequences from the test protein to MHC class II molecules is determined by in vitro analysis of the 9mer for binding to MHC class II.

41. The method of claim 32 where detection of binding of 9mer sequences from the test protein to MHC class II molecules is determined by positive identification of a 9mer sequence from a known helper T cell epitope with an identical match for residues 1, 4, 6, 7 and 9 in a 9mer sequence of the test protein.

42. The method of claim 33 where detection of binding of 9mer sequences from the test protein to MHC class II molecules is determined by positive identification of a 9mer sequence from a known helper T cell epitope with an identical match for residues 1, 4, 6, 7 and 9 in a 9mer sequence of the test protein.

43. The method of claim 34 where detection of binding of 9mer sequences from the test protein to MHC class II molecules is determined by positive identification of a 9mer sequence from a known helper T cell epitope with an identical match for residues 1, 4, 6, 7 and 9 in a 9mer sequence of the test protein.

44. The method as claimed in claim 32 wherein the immunogenicity of a test protein is analyzed by analysis of helper T cell epitopes.

45. The method as claimed in claim 32 where helper T cell epitopes within a test protein sequence are identified with the T cell epitopes being subsequently altered to remove T cell epitopes.

46. The method of claim 45 wherein alteration of T cell epitope sequences to remove a T cell epitope is achieved by conversion of the residue 1 in the T cell epitope from a hydrophobic to a non-hydrophobic residue.

47. A method for detection of non-helper T cell epitopes in a test protein sequence by analysis of 9mers from the test protein sequence by either of the following steps;
(a) Analysis of known non-helper T cell epitopes and identification of identical matches for residues 2, 3, 5 and 8 between one or more non-T cell epitopes and a 9mer sequence in the test protein sequence; OR
(b) Analysis and detection of non-binding by a 9mer sequence of the test protein to MHC class II molecules.

48. The method of claim 47 where matches with known non-helper T cell epitopes are determined by searching a database of non-helper T cell epitopes.


SEQUENCE LISTING (67 sequences)

SEQ ID NO: 1 - protein, 9 aa, Artificial
  9 amino acid test peptide which may be considered a possible T cell epitope
ADEFGHIKL

SEQ ID NO: 2 - protein, 9 aa, Artificial
  T cell epitope
AAAFAHIAL

SEQ ID NO: 3 - protein, 9 aa, Artificial
  T cell epitope
ADEAGAAKA

SEQ ID NO: 4 - protein, 9 aa, Artificial
  T cell epitope
GDEFGHIKL

SEQ ID NO: 5 - protein, 15 aa, Artificial
  Weak T cell epitope
GDKFVSWYQQGSGQS

SEQ ID NO: 6 - protein, 34 aa, Artificial
  Weak T-cell epitope
IKPEAPGCDASPEELNRYYASLRHYLNLVTRQRY

SEQ ID NO: 7 - protein, 15 aa, Artificial
  Weak T-cell epitope
QSISNWLNWYQQKPG

SEQ ID NO: 8 - protein, 16 aa, Artificial
  Strong T cell epitope
PKYRNMQPLNSLKIAT

SEQ ID NO: 9 - protein, 11 aa, Artificial
  Strong T cell epitope
TVFYNIPPMPL

SEQ ID NO: 10 - protein, 15 aa, Artificial
  Mutated T cell epitope
GDKGVSWYQQGSGQS

SEQ ID NO: 11 - protein, 15 aa, Artificial
  Mutated T-cell epitope
QSISNWGNWYQQKPG

SEQ ID NO: 12 - protein, 16 aa, Artificial
  Mutated T cell epitope
PKYRNGQPLNSLKIAT

SEQ ID NO: 13 - protein, 11 aa, Artificial
  Mutated T cell epitope
TVGYNIPPMPL

SEQ ID NO: 14 - protein, 13 aa, Artificial
  Peptide with fixed MHC contact residues
NWLRNYDQKQGAT

SEQ ID NO: 15 - protein, 13 aa, Artificial
  Peptide with fixed MHC contact residues
NWLEGYHQKIGAT

SEQ ID NO: 16 - protein, 13 aa, Artificial
  Peptide with fixed MHC contact residues
NWLLKYMQKFGAT

SEQ ID NO: 17 - protein, 13 aa, Artificial
  Peptide with fixed MHC contact residues
NWLPSYTQKWGAT

SEQ ID NO: 18 - protein, 13 aa, Artificial
  Peptide with fixed MHC contact residues
NWLYVYAQKRGAT

SEQ ID NO: 19 - protein, 13 aa, Artificial
  Peptide with fixed MHC contact residues
NWLNDYQQKEGAT

SEQ ID NO: 20 - protein, 13 aa, Artificial
  Peptide with fixed MHC contact residues
NWLGHYIQKLGAT

SEQ ID NO: 21 - protein, 13 aa, Artificial
  Peptide with fixed MHC contact residues
NWLKMYFQKPGAT

SEQ ID NO: 22 - protein, 13 aa, Artificial
  Peptide with fixed MHC contact residues
NWLSTYWQKYGAT

SEQ ID NO: 23 - protein, 13 aa, Artificial
  Peptide with fixed MHC contact residues
NWLAAYAQKAGAT

SEQ ID NO: 24 - protein, 13 aa, Artificial
  Peptide with fixed MHC contact residues
NWGRNYDQKQGAT

SEQ ID NO: 25 - protein, 13 aa, Artificial
  Peptide with fixed MHC contact residues
NWGEGYHQKIGAT

SEQ ID NO: 26 - protein, 13 aa, Artificial
  Peptide with fixed MHC contact residues
NWGLKYMQKFGAT

SEQ ID NO: 27 - protein, 13 aa, Artificial
  Peptide with fixed MHC contact residues
NWGPSYTQKWGAT

SEQ ID NO: 28 - protein, 13 aa, Artificial
  Peptide with fixed MHC contact residues
NWGYVYAQKRGAT

SEQ ID NO: 29 - protein, 13 aa, Artificial
  Peptide with fixed MHC contact residues
NWGNDYQQKEGAT

SEQ ID NO: 30 - protein, 13 aa, Artificial
  Peptide with fixed MHC contact residues
NWGGHYIQKLGAT

SEQ ID NO: 31 - protein, 13 aa, Artificial
  Peptide with fixed MHC contact residues
NWGKMYFQKPGAT

SEQ ID NO: 32 - protein, 13 aa, Artificial
  Peptide with fixed MHC contact residues
NWGSTYWQKYGAT

SEQ ID NO: 33 - protein, 13 aa, Artificial
  Peptide with fixed MHC contact residues
NWGAAYAQKAGAT

SEQ ID NO: 34 - protein, 9 aa, Artificial
  9mer MHC binding core
LNWYQQKPG

SEQ ID NO: 35 - protein, 12 aa, Artificial
  T cell epitope
GFGHEIGPLGEP

SEQ ID NO: 36 - protein, 9 aa, Artificial
  9mer T cell epitope core
LQHWSYPLT

SEQ ID NO: 37 - protein, 9 aa, Artificial
  Database T cell epitope
FLLTRILTI

SEQ ID NO: 38 - protein, 9 aa, Artificial
  Database T cell epitope
ILWEWASVR

SEQ ID NO: 39 - protein, 9 aa, Artificial
  Database T cell epitope
LSCAAGGRA

SEQ ID NO: 40 - protein, 9 aa, Artificial
  Database T cell epitope
FKGEQGPKG

SEQ ID NO: 41 - protein, 9 aa, Artificial
  Test peptide resulting from substituting contact residues onto a database T cell epitope
FQHTSILLI

SEQ ID NO: 42 - protein, 9 aa, Artificial
  Test peptide resulting from substituting contact residues onto a database T cell epitope
IQHESASLR

SEQ ID NO: 43 - protein, 9 aa, Artificial
  Test peptide resulting from substituting contact residues onto a database T cell epitope
LQHASGGLA

SEQ ID NO: 44 - protein, 9 aa, Artificial
  Test peptide resulting from substituting contact residues onto a database T cell epitope
FQHESGPLG

SEQ ID NO: 45 - protein, 9 aa, Artificial
  Control peptide with altered P1 residue
GQHWSYPLT

SEQ ID NO: 46 - protein, 9 aa, Artificial
  Control peptide with altered P1 residue
GQHTSILLI

SEQ ID NO: 47 - protein, 9 aa, Artificial
  Control peptide with altered P1 residue
GQHESASLR

SEQ ID NO: 48 - protein, 9 aa, Artificial
  Control peptide with altered P1 residue
GQHASGGLA

SEQ ID NO: 49 - protein, 9 aa, Artificial
  Control peptide with altered P1 residue
GQHESGPLG

SEQ ID NO: 50 - protein, 122 aa, Artificial
  N-terminus of CD20 antibody Leu16 heavy chain variable region (VH) sequence
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 51 - protein, 106 aa, Artificial
  N-terminus of CD20 antibody Leu16 light chain variable region (VL) sequence
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 52 - protein, 9 aa, Artificial
  T cell epitope in VH sequence of CD20 antibody Leu16
LVKPGASVK

SEQ ID NO: 53 - protein, 9 aa, Artificial
  T cell epitope in VH sequence of CD20 antibody Leu16
FKGKATLTA

SEQ ID NO: 54 - protein, 9 aa, Artificial
  T cell epitope in VH sequence of CD20 antibody Leu16
LTSEDSADY

SEQ ID NO: 55 - protein, 9 aa, Artificial
  Potential T cell epitope in VL sequence of CD20 antibody Leu16
ILSASPGEK

SEQ ID NO: 56 - protein, 9 aa, Artificial
  Potential T cell epitope in VL sequence of CD20 antibody Leu16
MDWYQKKPG

SEQ ID NO: 57 - protein, 9 aa, Artificial
  Database T cell epitope
LLSGSPAEK

SEQ ID NO: 58 - protein, 9 aa, Artificial
  Database T cell epitope
LDWYQKKPG

SEQ ID NO: 59 - protein, 9 aa, Artificial
  Database non-epitope 9mer
VVKPGASVK

SEQ ID NO: 60 - protein, 9 aa, Artificial
  Database non-epitope 9mer
FKGRVTLTA

SEQ ID NO: 61 - protein, 9 aa, Artificial
  Database non-epitope 9mer
LRSEDSAVY

SEQ ID NO: 62 - protein, 9 aa, Artificial
  Database non-epitope 9mer
TLSASPGEK

SEQ ID NO: 63 - protein, 9 aa, Artificial
  Database non-epitope 9mer
MAWYQQKPG

SEQ ID NO: 64 - protein, 117 aa, Artificial
  Heavy chain variable region of humanized A33 antibody
EVKLVESGGGLVKPGGSLKLSCAASGFAFSTYDMSWVRQTPEKRLEWVATISSGGSYTYYLDSVKGRFTISRDSARNTLYLQMSSLRSEDTALYYCAPTTVVPFAYWGQGTLVTVSA

SEQ ID NO: 65 - protein, 107 aa, Artificial
  Light chain variable region of humanized A33 antibody
ASTKGPSVFPLATSVGDRVSITCKASQNVRTVVAWYQQKPGQSPKTLIYLASNRHTGVPDRFTGSGSGTDFTLTISNVQSEDLADYFCLQHWSYPLTFGSGTKLEVK

SEQ ID NO: 66 - protein, 120 aa, Artificial [sequence omitted]

SEQ ID NO: 67 - protein, 107 aa, Artificial [sequence omitted]